(19) United States
(12) Patent Application Publication
(10) Pub. No.: US 2010/0136551 A1
(43) Pub. Date: Jun. 3, 2010

(54) MICROFLUIDIC PLATFORM AND RELATED METHODS AND SYSTEMS

(76) Inventors: Hansang CHO, Albany, CA (US); Brian R. BAKER, Livermore, CA (US); Luke P. LEE, Orinda, CA (US); Jane BEARINGER, Livermore, CA (US)

Correspondence Address: LLNL/Steinfl & Bruno;John H. Lee, Assistant Laboratory Counsel, Lawrence Livermore National Laboratory, L-703, P.O. Box 808, Livermore, CA, 94551, US

(21) Appl. No.: 12/576,209

(22) Filed: Oct. 8, 2009

Related U.S. Application Data
(60) Provisional application No. 61/104,627, filed on Oct. 10, 2008.
(60) Provisional application No. 61/175,822, filed on May 6, 2009.

(51) Int. Cl. C12Q 1/68; C12M 1/34; B29C 65/00; B32B 37/00

(52) U.S. Cl. 435/6; 435/287.2; 156/60; 156/62.2

(57) ABSTRACT

A microfluidic platform comprising one or more chambers connectable through microfluidic channels, and comprising a substrate presenting aptamer sensors detectable through Raman active molecules, and related methods and systems.

### CROSS REFERENCE TO RELATED APPLICATIONS

This application claims priority to U.S. Provisional Application entitled “Aptamer-based SERRS Sensor for Specific, Sensitive, and Stable Detection” Ser. No. 61/104,627, filed on Oct. 10, 2008 Docket No. IL-12034, and to U.S. Provisional Application entitled “Integrated Microfluidic Platform With Nanoplasmonic Aptasensor For On-Chip Label-Free Vegf Detection In Dynamic Tumor Microenvironment” Ser. No. 61/175,822, filed on May 6, 2009, the disclosure of each of which is incorporated herein by reference in its entirety. The Application may be further related to U.S. patent application entitled “Aptamer Based Sensors and Related Methods and Systems” Ser. No. to be assigned, filed on the same day of the present application with Docket No. IL12034, which is also herein incorporated by reference in its entirety.

### STATEMENT OF GOVERNMENT GRANT

The United States Government has rights in this invention pursuant to Contract No. Contract No. DE-AC52-07NA27344 between the U.S. Department of Energy and Lawrence Livermore National Security, LLC, for the operation of Lawrence Livermore National Security.

### TECHNICAL FIELD

The present disclosure relates to a microfluidic platform and related methods and systems.

### BACKGROUND

Microfluidic platforms provide a regulated microenvironment that is used to maintain biological samples, in particular cells by mimicking dynamic cellular microenvironments while stimulating specific cellular pathways by supplying chemicals.

A miniaturized environment created by the platform not only enables systematic characterization of cell responses in a high-throughput way, but also prevents human errors associated with repetitive tasks. Therefore, creating a microenvironment with microfluidics is considered an attractive way for performing parallel integration of cell culture and stimulus control in connection with assays or other applications in fields such as quantitative biomedicine.

An integrated microfluidic platform with a sensor is considered an attractive way for a long-term and real-time assessment of cellular secretion pathways under the spatial and temporal control of a chemically simulated tumor microenvironment.

### SUMMARY

Provided herein, is a microfluidic platform that allows in several embodiments detecting targets secreted from cells or another target producing sample located in the platform.

According to a first aspect, a microfluidic platform is described. The microfluidic comprises one or more chambers, configured to provide independent conditions within each of the one or more chambers, each chamber comprising a substrate presenting aptamer sensors. In particular, in several embodiments, the microfluidic chambers can be configured to provide a predetermined environment specific for a target producing material, such as cells, to be located within the microfluidic chambers. In several embodiments, the aptamer sensors are detectable by surface enhanced spectroscopy. In several embodiments the one or more microfluidic chambers are connected to an inlet reservoir and an outlet reservoir through microfluidic channels.

According to a second aspect a method to fabricate a chamber of a microfluidic platform is described. The method comprises: depositing carriers on a suitable substrate, the carriers suitable to attach aptamers, thus forming a carriers-substrate combination. The method further comprises bonding the carriers-substrate combination to a microfluidic structure open at its bottom, the carriers-substrate combination forming a bottom surface thus resulting in a microfluidic chamber. The method also comprises introducing the aptamers into the microfluidic chamber; and attaching the aptamers to the carriers in the chamber.

According to a third aspect, a method to detect target from a target providing material is described. The method comprises providing a microfluidic chamber comprising a surface presenting a target binding aptamer attaching a spectroscopic probe and placing one or more target producing material in the microfluidic chamber on the substrate. The method further comprises detecting a first spectrum of the surface presenting the target binding aptamer attaching the spectroscopic probe. The method also comprises stimulating the target producing material for a time and under conditions to allow production of the target from the target producing material and binding of the target with the target binding aptamer. The method further comprises detecting a second spectrum of the surface following contacting of the target, and comparing the first spectrum and the second spectrum.

The microfluidic platform and related methods and systems herein described can be used in several embodiments as a universal platform for quantitative study of biomarkers of interest such as growth factor and the related secretion and signaling.

Additionally, the microfluidic platform and related methods and systems herein described can be used in several embodiments for performing on-chip detection of a biomarker secreted by a cell while providing microenvironment for the secreting cells.

Furthermore, the microfluidic platform and related methods and systems herein described can be used in several embodiments as a dynamic tumor microenvironment by introducing various stimuli to isolated tumor cells with microfluidics and monitoring the interaction by detecting the secretome with integrated sensors.

Accordingly, the microfluidic platform and related methods and systems herein described can be used in several embodiments as a useful tool to quantitatively understand the tumor metastasis signaling pathway and discover drugs for cancer therapy.

Additionally, the microfluidic platform and related methods and systems herein described in several embodiments can easily accommodate numerous protein-specific aptamers with a variety of Raman probes for high throughput and multiplexed drug screening, biomedical diagnostics, and illicit drug or bio-agent detection.

The platforms, methods and systems herein described can be used in connection with applications wherein detection and/or analysis of a target molecule produced by biological samples such as cells or tissues are desired. Exemplary uses include but are not limited to medical application, biological analysis and diagnostics including but not limited to clinical applications. Additional applications include investigation of the effectiveness of drug candidates in a high-throughput way or multiplex diagnostics of biofluidics by using simple arrayed microfluidic channels without cell trapping structure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure.

FIG. 1 shows a schematic illustration of an integrated platform and of a detection method according to some embodiments herein described. In particular, FIG. 1A includes a photographic reproduction of a top view of a microfluidic platform according to an embodiment herein described where the inlet and outlet of the platform are indicated by arrow. FIG. 1B includes a photographic representation of a zoom-in view of a section indicated in FIG. 1A where the cell chambers of the platform are visible as white circles. FIG. 1C show a schematic representation of a top view of a chamber of the microfluidic platform indicated in FIG. 1B, wherein the molecules stimulating the cells are indicated with light grey dots and arrows and the molecule secreted by the cells are indicated in dark grey dots and arrows. FIG. 1D shows a schematic illustration of a cross sectional view along axis a-a′ of the cell chamber illustrated in FIG. 1C. FIG. 1E shows a schematic illustration of detection performed with a microfluidic platform according to an embodiment herein described.

FIG. 2 shows a schematic illustration of fabrication steps for manufacturing an integrated microfluidic platform according to some embodiments herein described. In particular FIG. 2A illustrates preparation of a substrate by sequential immobilization of aminopropyltriethoxysilane (APS), Gold Nanoparticles (GNP) and Poly-L-lysine (PLL). FIG. 2B illustrates fabrication of the microfluidic device by replication of a structure made of SU-8 on a wafer using polymeric structure. FIG. 2C illustrates bonding the substrate and the microfluidic device with UVO and immobilizing the aptamer on the substrate in the microfluidic substrate so obtained.

FIG. 3 shows a schematic illustration of a substrate optimization according to some embodiments herein described. In particular, in the illustration of FIG. 3 the optimization of the platform substrate is performed by variation of PLL molecular weight and incubation time. FIG. 3A shows representative images of a substrate, as PLL immobilization is optimized according to the experiments illustrated in FIG. 3B. Scale bars are 25 μm. FIG. 3B shows a diagram illustrating the effect of PLL immobilization at various molecular weights (x-axis) on the fluorescence intensity count (y-axis) detected by the platform in presence (+) or absence (−) of the substances indicated in the inset. FIG. 3C shows a diagram illustrating the effect of PLL immobilization at various incubation times (x-axis) on the fluorescence intensity count (y-axis) detected by the platform in presence (+) or absence (−) of the substances indicated in the inset. The inset in the diagram of Figure indicates presence of VEGF-Binding Aptamer (VBA), Vascular Endothelial Growth Factor (VEGF) and Fetal Bovine Serum (FBS) present (+) or absent (−) when a corresponding specific measurement illustrated in the chart is performed. Scale bars are 25 μm.

FIG. 4 shows characterization of stability of the sensor through integration steps according to some embodiments herein described. In particular FIG. 4 shows the characterization of bonding step's effect on the stability of APS, PLL, or VBA. FIG. 4A shows representative images for optimization of bonding protocol performed according to the experiments illustrated in FIG. 4B. Scale bars are 25 um. FIG. 4B shows a diagram illustrating the effect of UVO treatment after APS, PLL and VBA (x axis) on the fluorescence intensity count (y-axis) detected by the substrate. FIG. 4C shows a diagram illustrating the effect of plasma treatment after APS, PLL and VBA (x axis) on the fluorescence intensity count (y-axis) detected by the substrate.

FIG. 5 shows target detection by sensor in the integrated platform according to some embodiments herein described. In particular FIG. 5 shows VEGF detection in buffer solution in the integrated microfluidic platform. Scale bars are 500 μm. FIG. 5A shows representative a brightfield image before adding VEGF and fluorescent images from detection after 0, 20, and 60 min following the addition of VEGF (+). FIG. 5B shows a diagram plotting the normalized intensity detected (y-axis) versus time of VEGF addition (x-axis). FIG. 5C shows a diagram plotting the normalized intensity detected (y-axis) versus various VEGF concentrations (x-axis).

FIG. 6 shows target detection by sensor in the integrated platform according to some embodiments herein described. In particular, FIG. 6 shows VEGF detection from immobilized MCF-7 cells in integrated microfluidic platform. Scale bars are 500 um. FIG. 6A shows representative images from detection of a bright field image after 37 hours and fluorescence images after 0, 12, and 37 hr following the addition of culture media with estradiole as indicated. Scale bars are 500 μm. FIG. 6B shows a diagram plotting the normalized intensity detected (y-axis) versus distance of location where detection is performed from the cell location (x-axis) following addition of estradiole. FIG. 6C shows a diagram plotting the normalized intensity detected (y-axis) versus distance of location where detection is performed from the cell location (x-axis) in absence of estradiole.

FIG. 7 shows APS patterning on the aptasensor via porphyrin-based photocatalytic lithography. The first schematic shows that APS, which is originally uniformly coated, is selectively removed by exposure to reactive oxygen radical species emitted from porphyrin, or more generally, photosensitizer applied to a mask in close proximity to the silane layer and excited with visible light Subsequently, gold nanoparticle (GNP) solution, PLL solution, and aptamer solution are introduced on the APS-patterned substrate, resulting in selective patterning of aptamer sensor.

FIG. 8 shows the experimental results on aptamer patterning using masks having different arrayed-patterns. ‘Pattern 1’ has convex features of squares and lines and photosensitizer is coated on the surface in the region of squares and lines. ‘Pattern 2’ has concave features of squares and photosensitizer is coated on the surface excluding the region of squares. “No HP: NC” pattern has flat surface and is not coated with photosensitizer, “HP”. By bringing mask 1 with applied photosensitizers via pattern 1 in close proximity to the substrate surface coated with APS and exciting the mask with visible light, APS is removed in the region of squares and lines, resulting in aptasensor pattern outside the region. By performing the same procedure using pattern 2, APS is removed outside the region of squares, resulting in aptasensor pattern in the region. By using ‘No HP: NC’ pattern, APS is not removed and aptasensor is patterned on the entire surface.

FIG. 9 shows qualitative and quantitative target detection performed in saliva and serum from different patients according to some embodiments herein described. In particular in FIG. 9A representative images of VEGF detection performed on purified solutions including VEGF at 10 pM, 1 nM and 100 nM as indicated. FIG. 9B shows representative images of VEGF detection performed on saliva from four different individuals as indicated (saliva 1, saliva 2, saliva 3 and saliva 4). FIG. 9C shows representative images of VEGF detection performed on serum from four different individuals as indicated (serum 1, serum 2, serum 3 and serum 4). Scale bars are 25 μm.

FIG. 10 shows qualitative and quantitative target detection performed in saliva and serum from different patients according to some embodiments herein described. In particular, FIG. 10A shows a diagram illustrating a comparison of the fluorescent signals (y-axis) detected in samples of various origins (x-axis) as indicated. FIG. 10B shows a diagram illustrating a comparison of the fluorescent signals (y-axis) detected at various saliva concentrations (x-axis) from different individuals as indicated (#1 BC, patient 1; #2M: patient 2; #BMC: patient 3; #4C: patient 4).

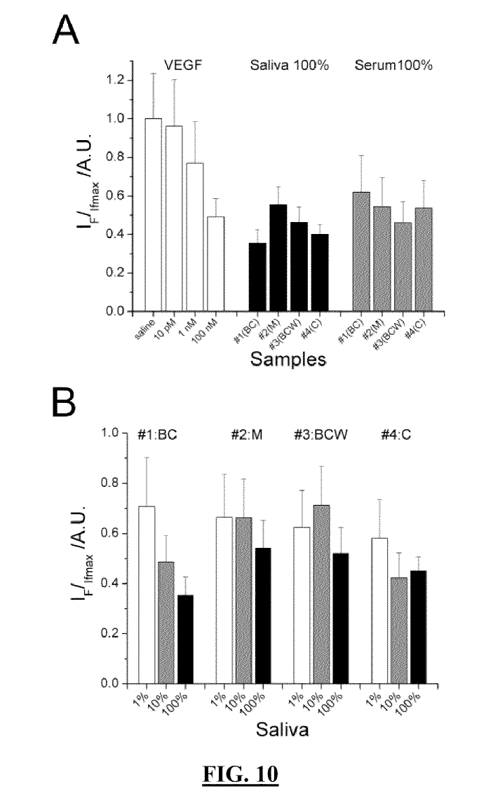

### DETAILED DESCRIPTION

Provided herein is a microfluidic platform for detection of a target present in a fluidic sample or solution or produced by a target producing material located inside the platform.

The term “microfluidic device” or “microfluidics” as used herein refers to a component and/or a system that manipulates fluid flow measured in nanoliters, picoliters, or femtoliters or channels and/or chambers that are generally fabricated in the micron or sub-micron scale. For example, the typical channels or chambers have at least one cross-sectional dimension in the range of about 0.1 microns to about 100 microns.

The term “platform” as used herein indicates a physical structure where analysis is realized. In the present disclosure, the microfluidic components can be included in an integrated microfluidic platform. As used herein, “integrated microfluidic platform” refers to a platform having two components: a microfluidic layor and a sensor substrate, which are physically and operably joined together to study on the interplays: chemical stimulus and cellular response of secretion. The components are fully or partially fabricated separately from each other and bonded after their fabrication. A microfluidic layor is a component that includes microfluidic chambers for cell trapping/culturing and microfluidic channels for cells/liquid distribution. A sensor substrate is a component that includes gold nano materials coated with charged molecules and aptamers conjugated with probe molecules.

The microfluidic systems can also be provided in a modular form. The term “modular” describes a system or device having multiple standardized components for use together, wherein one of multiple different examples of a type of component may be substituted for another of the same type of component to alter the function or capabilities of the system or device; in such a system or device, each of the standardized components being a “module”.

The term “detect” or “detection” as used herein indicates the determination of the existence, presence or fact of a target or signal in a limited portion of space, including but not limited to a sample, a reaction mixture, a molecular complex and a substrate including a platform and an array. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through visually detectable signals: spectra or images from a target of interest or a probe attached to the target.

The term “target” as used herein indicates an analyte of interest. The term “analyte” refers to a substance, compound or component whose presence or absence in a sample has to be detected. Analytes include but are not limited to biomolecules and in particular biomarkers. The term “biomolecule” as used herein indicates a substance compound or component associated to a biological environment including but not limited to sugars, aminoacids, peptides proteins, oligonucleotides, polynucleotides, polypeptides, organic molecules, haptens, epitopes, biological cells, parts of biological cells, vitamins, hormones and the like. The term “biomarker” indicates a biomolecule that is associated with a specific state of a biological environment including but not limited to a phase of cellular cycle, health and disease state. The presence, absence, reduction, upregulation of the biomarker is associated with and is indicative of a particular state. The term “biological environment” refers to any biological setting, including, for example, ecosystems, orders, families, genera, species, subspecies, organisms, tissues, cells, viruses, organelles, cellular substructures, prions, and samples of biological origin. Exemplary targets comprise molecular targets such as small molecules, proteins, nucleic acids, and also cells, tissues and organisms.

In several embodiments, the target to be detected is in a fluidic sample or a solution The term “sample” as used herein indicates a limited quantity of something that is indicative of a larger quantity of that something, including but not limited to fluids from a biological environment, specimen, cultures, tissues, commercial recombinant proteins, synthetic compounds or portions thereof. The term “solution” as used herein comprises a single-phase or multiple phase liquid system, also including colloids and suspensions. Exemplary solutions include homogeneous mixture composed of two or more substances, where typically a solute is dissolved in another substance, known as a solvent. Additionally exemplary solutions in the sense of the disclosure include non-homogeneous mixtures such as chemical mixture in which one substance is dispersed evenly throughout another (colloids) and heterogeneous fluid containing solid particles that are sufficiently large for sedimentation (suspensions).

In some embodiments, the target to be detected is provided in the platform by a target producing material, which in the sense of the present disclosure comprise any substance, biological or non biological, that is capable of producing an analyte of interest under appropriate conditions.

In some embodiments the target producing materials is formed by or comprises cells. In particular in several embodiments the cells can be formed by cell lines such as MCF-7 is a breast cancer cell line. This cell line retained several characteristics of differentiated mammary epithelium including the ability to process estradiol via cytoplasmic estrogen receptors and the capability of forming domes. No additional treatment is required.

In some embodiments, the target producing material is formed by or comprises target producing materials other than cells. For example, drug-delivery devices (such as liposomes, biodegradable microspheres, and additional products identifiable by a skilled person) can be used in addition, in combination or in place of cells. In particular, in exemplary embodiments where drug delivery devices are comprised as target producing material the device could be used to monitor the release of the drug from the drug delivery device under different conditions. In those embodiments, the platform can be used for example to detect drugs release (e.g. hypericin, and emodin on aqueous silver colloid as well as other small molecules) and possibly also corresponding interaction with cells also located on a same chamber.

In several embodiments, the platform comprises one or more chambers with each chamber configured to provide independent conditions with respect to another and/or other environment within the platform. This configuration allows performing target detection from a sample, solution and/or target producing material located within a microenvironment that is controllable by a skilled user.

In particular, in several embodiments, the microfluidic chambers can be configured to provide an isolated environment for target producing material (e.g. cells) to be located within the microfluidic chambers. For example, in several embodiments, the microfluidic platform provides perfused media or solution within the platform and in particular within the chambers, to control chambers conditions (e.g. to maintain cells alive and viable should the chamber include cells).

Additionally, in some embodiments, the one or more chambers are connectable through microfluidic channels to an inlet reservoir and an outlet reservoir. In some embodiments the platform is transparent for optical detection. In several embodiments the one or more chambers comprise a substrate on which aptamer sensors are located.

In particular target detection in the microfluidic platform is performed typically by aptamer sensors attaching a probe. The term “aptamers” as used here indicates oligonucleic acid or peptide molecules that are capable to bind a specific target.

The terms “oligonucleic acid”, “nucleotidic oligomer” or “oligonucleotide” as used herein, indicate an organic polymer composed of two or more monomers including nucleotides, nucleosides or analogs of three or more residues typically of 100 nucleotides or less. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group.

The terms “peptide” and “oligopeptide” as used herein indicate an organic linear, circular, or branched polymer composed of two or more amino acid monomers and/or analogs thereof with 50 or less amino acid monomers. As used herein the term “amino acid”, “amino acidic monomer”, or “amino acid residue” refers to any of the twenty naturally occurring amino acids, non-natural amino acids, and artificial amino acids and includes both D an L optical isomers. In particular, non-natural amino acids include D-stereoisomers of naturally occurring amino acids (these including useful ligand building blocks because they are not susceptible to enzymatic degradation). The term “artificial amino acids” indicate molecules that can be readily coupled together using standard amino acid coupling chemistry, but with molecular structures that do not resemble the naturally occurring amino acids. The term “amino acid analog” refers to an amino acid in which one or more individual atoms have been replaced, either with a different atom, isotope, or with a different functional group but is otherwise identical to original amino acid from which the analog is derived. All of these amino acids can be synthetically incorporated into a peptide or polypeptide using standard amino acid coupling chemistries.

In particular, aptamers un the sense of the present disclosure comprise single-stranded (ss) oligonucleotides and chemically synthesized peptides that have been engineered through repeated rounds of in vitro selection, or equivalent techniques identifiable by a skilled person, to bind to various targets.

The term “sensor” as used herein indicates a device that measures a physical quantity and converts it into a signal which can be read by an observer or by an instrument. For accuracy, sensors need to be calibrated against known standards. Accordingly, the wording “aptamer-based sensor”, aptasensor, or aptamer beacon used herein indicate a sensor that can be used to capture a target exploiting the affinity of aptamer to the target and that can be detected using techniques identifiable by a skilled person upon reading of the present disclosure.

The term “probe”, “label” and “labeled molecule” as used herein as a component of a complex or molecule referring to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “signal” or “labeling signal” as used herein indicates the signal emitted from the label that allows detection of the label, including but not limited to radioactivity, fluorescence, chemiluminescence, production of a compound in outcome of an enzymatic reaction and the like.

In several embodiments, aptamers change their secondary structures depending on ionic environments and in the presence of molecules having high affinity. For example, a thrombin-binding aptamer forms ‘Guanine Quadruplex’ or ‘G-Quadruplex’ in the presence of a thrombin protein or high concentration of potassium. [Ref. 1-9]

Exemplary aptamer-based sensors of the present disclosure comprise aptamer based sensor developed in connection with any one of the detection techniques indicated above and in particular with surface enhanced spectroscopy to detect interaction between the aptamer and an analyte of interest (e.g. via single or multiple binding events) and subsequent detection of the labeling signal changes with a complex including a Raman or fluorescent probe which is then detected through surface enhanced Raman spectroscopy or surface enhanced fluorescence microscopy.

In particular in several embodiments, the aptamer-based sensors of the present disclosure are detectable through spectroscopic detection techniques such as SERRS, SERS or SEF (herein collectively Surface Enhanced Spectroscopy). The term “Surface Enhanced Spectroscopy” as used herein indicates signal enhancement techniques where signal detection from corresponding spectroscopic probes is performed in connection with a metal surface. Exemplary spectroscopic techniques suitable to detect aptamer based sensor herein described comprise including Surface-Enhanced Resonance Raman Spectroscopy (SERRS), Surface-Enhanced Raman Spectroscopy (SERS), Surface-Enhanced Fluorescence (SEF), Surface-Enhanced Infrared Absorption (SEIRA), Surface-Enhanced Hyper-Raman Scattering (SEHRS), Surface-Enhanced Coherent Anti-Stokes Raman Scattering (SECARS), and additional techniques identifiable by a skilled person.

The term “spectroscopic probe” as used herein indicates any substance that is suitable to be detected based on an interaction between a radiation and the substance through a spectroscopic instrument. Exemplary spectroscopic probes comprise Raman probes and fluorescence probes. The terms “Raman active molecule” or “Raman probe” as used herein refer to a molecule capable having a polarization-dependant vibrational mode excited by an incident light. The vibrational energy stored in the molecule is transformed into a scattering light corresponding to a specific frequency. In particular, detected signals emitted by Raman probes can take the form of Raman spectra. Accordingly, in Raman spectra for a certain Raman probe, each peak represents the vibrational frequency corresponding to resonance energy of the functional groups in the Raman probe as detected. Therefore, Raman spectra are intrinsic properties of the molecules such as a “molecular fingerprint” to identify the molecule without need to use of any additional labels.

In some embodiments, Raman probes suitable to be included in the aptamer-based sensors herein described comprise Raman-active molecules having polarization-dependant translational and/or rotational modes. Exemplary Raman probes suitable to be used for aptamers based sensors herein described comprise Trans-1,2 bis-(4-pyridyl)ethylene (BPE), Cy-3, Cy-3.5, Cy-5, Cy-5.5, Cy-7, Rhodamine 6G (R6G), methylene blue (MB), 5-carboxyfluorescein or 6-carboxyfluorescein (FAM), N,N,N′,N-tetramethyl-6-carboxyrhodamine (TAMRA), 6-carboxy-4,7,2′,7′-tetrachlorofluorescein (TET), 6-carboxy-Xrhodamine (ROX), (3-(5,6,4′,7′-tetrachloro-5′-methyl-3′,6′-dipivaloylfluorescein-2-yl)-propanamidohexyl-1-O-(2-cyanoethyl)-(N,N-diisopropyl)) Yakima yellow®, 6-(((4(4,4-difluoro-5-(2-thienyl)-4-bora-3a,4a-diaza-s-indacene-3-yl)phenoxy)acetyl)amino)hexanoic acid (BODIPY TR-X) and additional probes identifiable by a skilled person upon reading of the present disclosure.

The term “fluorescent probe” as used herein indicates any substance that is detectable through emission of a visible light by the substance following absorption by the same substance of light of a differing, usually invisible, wavelength. Exemplary fluorescent probes suitable in the aptamer-based sensors herein described comprise Cy-3, Cy-3.5, Cy-5, Cy-5.5, Cy-7, Rhodamine 6G (R6G), methylene blue (MB), TAMRA and additional probes identifiable by a skilled person.

In several embodiments, the microfluidic platform comprises surface Plasmon and aptamer-based sensors operatively connected to an array configured to host a target producing material.

In certain embodiments, the platform includes Local surface Plasmon resonance (LSPR) LSPR is an approach to enhance surface Plasmon locally, resulting in dramatic enhancement of the signal of a Raman or fluorescence molecule in close proximity to metal surfaces where the strong surface plasmon is locally created or concentrated [1,2]. In order to provide LSPR effect in the platform, novel metal (gold or silver) spherical particles are used or aggregated/patterned on the substrate as nanostructures with sharp tips or small gaps on the nano or subnano scale.

In several embodiments, spectroscopic probes used in the aptamer-based sensor of the present disclosure enable an enhancement factor e as much as 10¹⁴-10¹⁶, which allows the technique to be sensitive enough to detect single molecules.

In several embodiments, the device can be used with any detectable aptamer able to bind a target of interest. Aptamers can be designed to bind to almost any kind of target molecule, so the device can be configured to give response to almost any kind of target molecule.

In particular, in several embodiments, the microfluidic platform is composed of a microfluidic channel to provide cellular environments and a glass substrate coated or patterned with aptamer sensors, which are fabricated separately excluding an immobilization process for aptamer complex. The aptamer complex is introduced through microfluidics for immobilization after bonding the microfluidic channel and the glass substrate.

The platform can include either SERRS (or SERS) sensor or SEF (surface enhanced fluorescence) sensor, which commonly exploits aptamers for target recognition and surface Plasmonics for an amplified readout.

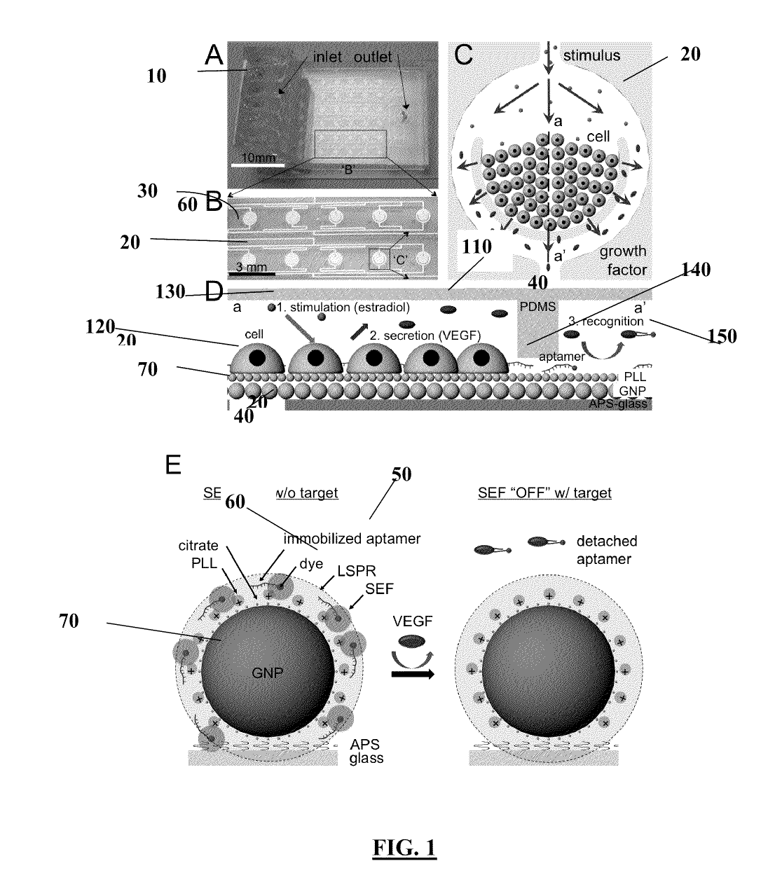

As shown in FIG. 1, the microfluidic platform (10) comprises one or more chambers (20) connectable through microfluidic channels (30). The chambers (20) have a substrate (40) on which aptamer sensors are located. Each aptamer sensor comprises an aptamer (50) attached to a Raman probe or dye (60).

The substrate (40) can be, for example, a glass substrate and the aptamer sensors, in their initial condition, can be physisorbed on surfaces where carriers such as golden nanoparticles (GNP) (70) are located. The term “carrier” as used herein indicates a material that is capable to attach and support aptamers on a surface while maintaining and possibly improving the detectability of the surface by one of the methods herein described. In some embodiments, a combination of carriers is provided on the surface to support the aptamers used for detection within a chamber.

Additional carriers comprise various metal nanoparticles possibly comprising agents favoring attachment of an aptamer such as polylysine. The shape and size of the metal nanoparticles is selected in view of the desired enhancement because these factors influence the peak and amplitude of surface Plasmon. Not only spherical particles can be prepared but a variety of shapes including cubes, prisms, rods, octahedral, depending on reaction conditions and surface-active agents. A skilled person will be able to identify the desired shape and size of metal particles to be used in connection with a surface herein described using, for example, analytical model or numerical calculation. In several embodiments, the metal is formed by gold and/or silver nanoparticles that have surface plasmonic resonance peaks in visual wavelength range and the surfaces can be easily modified by conjugating functional chemicals. Additional nanoparticles comprise bimetal nanoparticles of silver and gold, mixed colloids of Ag or gold with catalytically important palladium and other nanoparticles described in U.S. Pat. No. 6,149,868 and in Ref. [37] each incorporated herein by reference in its entirety.

In the illustration of FIG. 1, each chamber is isolated from the other so that the flow passing through a first chamber never passes over another chamber thus minimizing any contamination and/or cross-linking. Therefore in this embodiment the arrayed chambers can be used to perform a study where several targets and/or systems are analyzed independently.

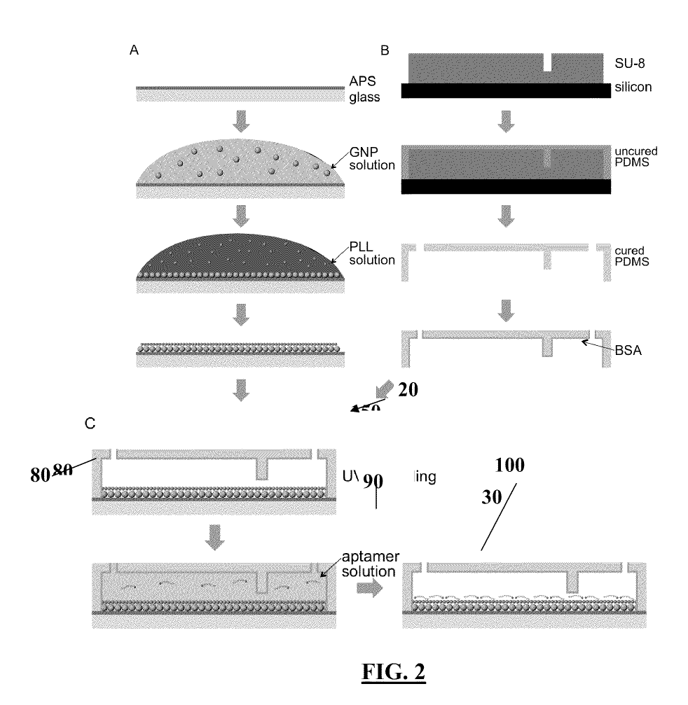

The fabrication process of the chambers (20) of the microfluidic platform (10) is shown in FIG. 2 and is such that the golden nanoparticles (GNP) and poly-L-lysine (PLL) are deposited on an APS-coated glass substrate (inset A) and the substrate is then bonded (e.g., UVO bonding, inset C) with a microfluidic structure (inset B) open at the bottom to form the bottom surface (80) of the microfluidic chamber (20). As soon as this is done, an aptamer solution (90) is flown into the microfluidic chamber (20) and the aptamers (100) are then physisorbed to the surface of the GNPs through the PLL layer with electrostatic force.

Therefore, according to an embodiment of the disclosure, the bottom surface (80) of the microfluidic chambers (20) comprises an APS-coated glass substrate on which GNPs and PLL are disposed, with aptamer sensors physisorbed on the top of PLL.

Substances, such as PLL and APS can be provided on the substrate to facilitate binding of the aptamers, and are herein collectively identified as aptamer-substrate binders. In particular, the properties of molecules such as poly-L-lysine (PLL) modulate binding of the aptamer to the substrate when the aptamer is introduced into the device. The optimal conditions for PLL immobilization can be adjusted in view of the specific aptamer used. If the size of the aptamer is smaller or larger or if a peptide-based aptamer is employed, different immobilization conditions can be needed for optimal performance, which are identifiable by a skilled person upon reading of the present disclosure (see Example 2).

In several embodiments, the substrate can further be subjected to a treatment suitable to create OH− or H+ functional groups on glass surface for enhancing bonding between microfluidic components such as microfluidics made of PDMS or glass. As to create the groups, glass surface is exposed to oxygen plasma or UV and ozone (UVO), which degrades molecules composing aptasensors. The stability of the aptasensor was evaluated by treating both methods on the surface after each step to evaluate the stability of each molecule. The results show that aptamer is seriously degraded by both of oxygen plasma and UVO but more than half of APS and PLL remained stable after UVO treatment. Based on the evaluation, UVO process was included after PLL and before aptamer coating. Additionally, in several embodiments, UV-SERS don't need “coinage metals” to cover Ag/Au/Cu/Ni and combinations.

The platform exemplified in FIG. 1, can be used for target detection by exploiting the ability of the aptamers to bind the targets and to be detectable In particular, in some embodiments, a first spectrum of the surface presenting target binding aptamers and attaching a probe can be performed and compared with a second spectrum of the same surface after contacting the target with the target binding aptamer attaching the probes presented on the surface.

The term “spectrum” as used herein indicates a representation (in particular a graphic representation such as a plot) of wavelengths reflected from a surface, which varies in function of chemical and/or physical properties (e.g. irregularities, atomic composition and/or molecular composition) of the surface.

The term “present” as used herein with reference to a compound or functional group indicates attachment performed to maintain the chemical reactivity of the compound or functional group as attached. The term “attach” or “attached” as used herein, refers to connecting or uniting by a bond, link, force or tie in order to keep two or more components together, which encompasses either direct or indirect attachment where, for example, a first molecule is directly bound to a second molecule or material, or one or more intermediate molecules are disposed between the first molecule and the second molecule or material. The term “bind”, “binding”, “conjugation” as used herein indicates an attractive interaction between two elements which results in a stable association of the element in which the elements are in close proximity to each other. If each element is comprised in a molecule the result of binding is typically formation of a molecular complex. Attractive interactions in the sense of the present disclosure includes both non-covalent binding and, covalent binding. Non-covalent binding as used herein indicates a type of chemical bond, such as protein-protein interaction, that does not involve the sharing of pairs of electrons, but rather involves more dispersed variations of electromagnetic interactions. Non-covalent bonding includes ionic bonds, hydrophobic interactions, electrostatic interactions, hydrogen bonds, and dipole-dipole bonds. Electrostatic interactions include association between two oppositely charged entities.

In several embodiments, detection can be performed by providing a microfluidic chamber comprising a substrate on which target binding aptamers are presented and placing one or more target producing material in the microfluidic chamber on the substrate. The method further comprises detecting a first surface enhanced spectrum a target binding aptamer, with the target binding aptamer specific for the target and attaching a spectroscopic probe. The method further comprises stimulating the target producing material for a time and under conditions to allow production of the target from the target binding material and binding of the target with the target binding aptamer. The method can also comprises detecting a second surface enhanced spectrum of the target binding aptamer attaching the spectroscopic probe following contacting of the target binding aptamer with the target and comparing the first spectrum and the second spectrum.

In particular, detection of a first enhanced spectrum and second enhanced spectrum can be performed in connection with use of the aptamer based sensor described in the related U.S. patent application entitled “Aptamer Based Sensors and Related Methods and Systems” Ser. No. to be assigned, filed on the same day of the present application with Docket No. IL12034, incorporated herein by reference in its entirety. In particular, in several embodiments, when the aptamer based sensors of application IL12034 are used in the platform, the aptamer attaching the spectroscopic probe presented on the surface detaches from the surface, following binding with a corresponding specific target, which results in a modified spectrum of the surface. In particular, in several embodiments response of Raman spectroscopy performed on the surface (e.g. GNPs distributed over silica or glass substrate) is dependent on the amount of aptamers physisorbed on the surface. Therefore, in those embodiments the response obtained after target stimulation can be indicative not only of presence or absence of aptamer binding, but also of the amount of targets that binds the aptamers.

In an exemplary embodiment illustrated in FIG. 1D, targets (110) are generated or secreted by one or more cells (120) placed on the bottom surface of the microfluidic chambers. Secretion of targets can be obtained through stimulation of the cells with molecules (130), such as estradiol. If, for example, a stimulating molecule (130) is such to elicit secretion of VEGF (110) from a stimulated cell (120), and aptamers (140) adapted to bind to VEGF (110) are physisorbed on the surface of the GNPs (70), such aptamers bind (150) to the VEGF and detach from the surface of the GNPs in an amount dependent on the amount of VEGF secreted by the cells (120). When Raman spectroscopy is performed on the GNPs, a response proportional to the secreted VEGF is obtained.

In embodiments where the target producing material is formed by substances other than cells, the material, (e.g. a drug delivery device) can be placed in the same region shown occupied by cells in FIG. 1C. The device could be used to monitor the release of the drug from the drug delivery device under different conditions, if the immobilized aptamer is one that will specifically recognize and bind to the drug.

In particular, in embodiments where the target is produced by a target producing material the platform substrate is typically patterned. In particular, in several embodiments patterning aptamer sensors can be performed on the micron or nanometer scale through techniques such as traditional lithography, porphyrin-based photocatalytic lithography, contact printing or dip pen lithography and additional techniques identifiable by a skilled person.

In several embodiments, the platform is configured to provide arrayed chambers in micrometer or millimeter scale, where systematic study can be achieved in chambers set up to provide a same or different environment. Therefore, in embodiments where chambers are set up to provide identical conditions a single platform allows performance of high throughput detection where systematical and statistical analysis can be carried out with a high speed and minimized errors.

Therefore, the arrangement of FIG. 1 can be used as a detection mechanism where one among stimulating molecules, stimulated cells, targets, aptamers and/or other components are unknown and is recognized through the above described detection method.

In several embodiments, right after trapping cells in arrayed chambers in the platform, fluorescence (Raman) signals are measured from each of chambers for a signal of baseline. Every hour during a culturing period, the fluorescence signals are measured from the same chambers for time-dependant monitoring.

In several embodiments the platform can be used for on-chip and real-time detection of biomolecules secreted from cells cultured in the platform. A media containing 10% FBS is perfused into the platform at the flow rate of 0.5 μL/min during experiments.

In particular, several embodiments in the absence of targets, dye conjugated aptamers (e.g. Cy3-conjugated VEGF binding aptamer (VBA) is immobilized on 80 nm gold nanoparticle (GNP) surfaces and a baseline intensity is observed as local surface plasmon resonance (LSPR) induces surface enhanced fluorescence (SEF) of Cy3. Secreted VEGF, induced by estradiole, interacts with the aptamer resulting in displacement of the aptamer from the GNP surface and a subsequent decrease in fluorescence intensity by displacing Cy3 from the LSPR region [4].

In several embodiments, the microfluidic platform herein described allows detection of targets at concentration ranging from about 100 nM to about 1 nM. (see Example 5).

In several embodiments, the microfluidic platform herein described allows sensitive detection of targets (e.g. 1 nM) even under harsh conditions. In particular in several embodiments, the aptamer-based sensor can be used for detection in conditions where protein or protein based sensor are usually degraded but aptamers are not (harsh condition). Those conditions include for example, detection from blood, where proteinases are present, which degrades protein-based antibodies in a short time. Also included is detection performed for a time and at a temperature that are usually associated with degradation of a protein which comprise exposure at high temperature for a short a amount of time or a lower temperature (e.g. room temperature) for a longer amount of time (e.g. several hours). High temperatures and low temperature are identifiable by a skilled person based on the specific protein. Modified aptamers are known to be stable even above melting temperature of several proteins.

In several embodiments, the microfluidic platform herein described can be used to quantitatively detect targets and in particular biomarkers secreted from cells. In particular to perform the quantitative detection the nanosensor was patterned in an array format by selectively removing a linking molecule using photocatalytic lithography.

In particular, in several embodiments, in order to achieve quantitative detection of a specific target molecule, a calibration curve can be created where the response of the device is measured after exposing the device to several different concentrations of the target molecule and measuring the response. Accordingly, in embodiments when an unknown amount of the target molecule is produced by the target producing materials, the measured response can be compared to the calibration curve and the unknown concentration can be calculated.

In several embodiments qualitative detection can be performed following a comparison between control results, e.g. signals from an aptamer sensor without exposure to a target and with exposure to a target extremely high concentration.

In particular, in several embodiments, the platform can provide a measurable response in absence of target. By measuring this background response level, when the device is operated in absence of target, a threshold value can be established. Accordingly, when unknown concentrations (which can also be zero) are produced by the target producing materials, the device response can be compared to the threshold value, such that any value above the threshold will give a qualitative determination that the target molecule is present.

In several embodiments, target detection can be performed while varying the microenvironment for the cells or other target producing material. By introducing variant concentration and kinds of stimulus to a signal platform in an array format, the cellular response can be analyzed quantitatively in a high-throughput. Several of those embodiments provide an advantage over certain current detection methods based on antibodies, which show instability under long-term culturing conditions (37° C.), and require the extraction of conditioned medium thus precluding monitoring of cellular behavior in dynamic tumor microenvironments.

In several embodiments, one major advantage of this device is the ability to control the microenvironment experienced by the target producing materials to investigate the affect of the amount of target material produced/released. For example, if the target producing material is live cells, the microenvironment can be adjusted by addition of a compound. A skilled user will be able to determine if the amount of target material produced or released increases or decreases upon addition of the compound. In another example, if the target producing material is a drug delivery device, the microenvironment solution conditions (such as acidity) can be changed to mimic different conditions inside the body. In particular, a skilled user will be able to determine if the amount of target material produced or released changes, and be able to characterize the drug delivery properties.

In particular in several embodiments it is possible to investigate the correlation between introduced stimulus and cellular secretion response. The stimulation can be varied with concentration and period. The cellular response can be monitored in real time during the entire culturing period.

Stimulation of a target producing material in the microfluidic platform herein described can be performed with several methods identifiable by a skilled person in view of the target material produced, which include but are not limited to addition of single or multiple compounds. In this connection, in exemplary embodiments where the stimulation is the addition of a single compound to the microenvironment, the concentration of the single compound can be set to several different values, and the response of the target producing materials can by characterized at several different concentrations. In other exemplary embodiments, where stimulation involves the addition of multiple compounds to the microenvironment, different ratios of the concentrations of the compounds can be used.

In several embodiments, the platform can be used to detect biomarkers secreted by cells and to analyze their properties and/or activities. In particular, the platform can be used to detect cancer markers secreted from tumor calls. In those embodiments the platform can be configured to provide a dynamic tumor microenvironment by introducing various stimuli to isolated tumor cells with microfluidics and monitors the behavior by detecting the secretome with an integrated nanosensor. For example, in embodiments where activation of VEGF secretion pathway is desired, such activation can be performed by subjecting the cells to deficiency of nutrition and oxygen or by treating the cells with an activator, such as estrodiole. In other exemplary embodiments where inhibition of VEGF secretion pathway is desired, such inhibition can be achieved by contacting the cells with an inhibitor, interferon-α regulating pathways that are related to proliferation. Therefore, in several embodiments a pathway can be activated or inhibited by culturing cells with media deficient of nutrition or supply of low concentration oxygen, or media including stimulus. A skilled person will be able to identify the specific compound and/or conditions that are to be applied to a certain cell to trigger the desired response to be detected and/or analyzed.

In several embodiments, the microfluidic platform herein described can be used for studying antiangiogenic agents, and other cell produced factors, which are important in clinical prognosis. In other embodiments, the microfluidic platform herein described can be used for high-throughput screening of drug candidates, such as for example the ones that inhibit VEGF secretion.

In particular in several embodiments, aptasensors are patterned on a bottom substrate in an array format. On top of the substrate, a microfluidics with arrayed channels or a cover with arrayed holes covers. Solutions of drug candidates are introduced or pipetted through the microfluidics or the cover, respectively and then the effectiveness of the candidates is evaluated by monitoring binding events with aptasensor.

In some embodiments, the microfluidic platform herein described can be used to analyze early processes and the effect of microenvironment on metastatic signaling pathways and, in turn, eventually in clinical research. For example, the platform can be used to study cancer metastases commonly found in the lymphatic system and circulatory system' because stimulation of tumor lymphangiogenesis and tumor blood angiogenesis require the interplay of several tumor-derived growth factors. In some of those embodiments, the blood and lymph associated cells then secrete growth factors (secretome) which stimulate motility and invasion of tumors. Multiple growth factors: vascular endothelial growth factor (VEGF), basic fibroblast growth factor (bFGF), platelet derived growth factor (POGF), and transforming growth factor (TGF-˜) interrupt angiogenic dormancy, promote neovascularization, and enhance the proliferation of tumor cells. Thus, monitoring the level of these secreted growth factors will provide an approach to understand tumor metastasis. Although there are many hypotheses from clinical and laboratory data on the mechanism of growth factors in angiogenesis and metastasis, there is not yet any definitive and quantitative evidence for their efficacy, Antibody-based detection methods are limited due to their instability under long-term culturing conditions (3rC). Additionally, detection of growth factors using antibodies requires the extraction of conditioned medium, which precludes monitoring of cellular behavior in dynamic tumor microenvironments. Additional molecules detectable with the platform comprise glucose, small molecules, proteins and additional biomarkers identifiable by a skilled person.

Further details concerning the platforms and related methods and systems, and generally manufacturing of the various components, can be identified by the person skilled in the art upon reading of the present disclosure.

### EXAMPLES

The methods and systems herein disclosed are further illustrated in the following examples, which are provided by way of illustration and are not intended to be limiting.

In the following examples, an exemplary integrated nanoplasmonic aptasensor (aptamer sensor) is described within a microfluidic device for on-chip label-free detection of secreted growth factor under the spatial and temporal control of a simulated tumor microenvironment. In particular, the nanoplasmonic aptasensor of the examples was realized by utilizing highly specific aptamer displacement upon target binding and monitoring signal change of SEF (surface-enhanced fluorescence) resulting from the displacement. Here, we verified that the nanosensor could detect a cancer marker, VEGF (vascular endothelial growth factor). The VEGF was detected by monitoring the SEF signal decrease of a Cy3 conjugated with VEGF binding aptamer (V8A). Upon the VEGF binding to the V8A immobilized on the gold nanopanides'surface, the scallered spectra diminished in intensity as the V8A was displaced from the gold surface. The nanosensor realized VEGF detection down to 1 nM in purified solution and showed the distinguishable signal change in MCF-7 cells stimulated by estrodiole.

### Example 1

### Fabrication of a Microfluidic Platform

An integrated platform was fabricated following a procedure schematically illustrated in FIG. 2.

In the illustration of FIG. 2, APS, GNP, and PLL are sequentially immobilized on a glass surface (see in particular FIG. 2A). In particular, the glass slide was thoroughly rinsed with acetone and isopropyl alcohol (IPA) sequentially and then modified with amino-terminal group by immersion in APS (10% v/v with IPA) for 10 minutes followed by rinsing with IPA and drying with N₂ gas. A 100 μL GNP solution was spotted on the modified slide (2 cm×3 cm) defined by a PDMS membrane and incubated at room temperature for an hour. Unfixed GNPs were removed by rinsing the substrate three times with 10 μL of DI water. 70 kDa PLL in buffer solution was then immobilized on the GNP-substrate by incubating for 5 minutes at room temperature. The unbound PLL were subsequently removed via repeated rinsing five times with 10 μL buffer.

The microfluidic device is then fabricated by replicating structure made of SU-8 on a wafer using PDMS as schematically shown in FIG. 2B. In particular, a mold of SU-8 was fabricated with standard lithography. PDMS microfluidics was fabricated by replicating negative pattern of SU-8. The fabricated PDMS was coated with 5% w/v BSA for 1 hour at room temperature to avoid adsorption of aptamer and target protein to PDMS surface.

Both of BSA-coated PDMS microfluidics and PLL-GNP-APS coated glass surface were exposed to UVO for 3 minutes and instantly contact for bonding. The bonded platform was cured more than 1 hour at room temperature for the surface recovery after the UVO treatment. After recovery, an aptamer in buffer at the concentration of 100 nM were immobilized on the PLL-covered GNP surface by perfusion at the rate of 1 uL/min for 1 hour above melting temperature (i.e. 70° C.). The unbound aptamers were subsequently removed via rinsing with buffer of 1 mL.

### Example 2

### Platform with Optimized of Signal to Noise Level

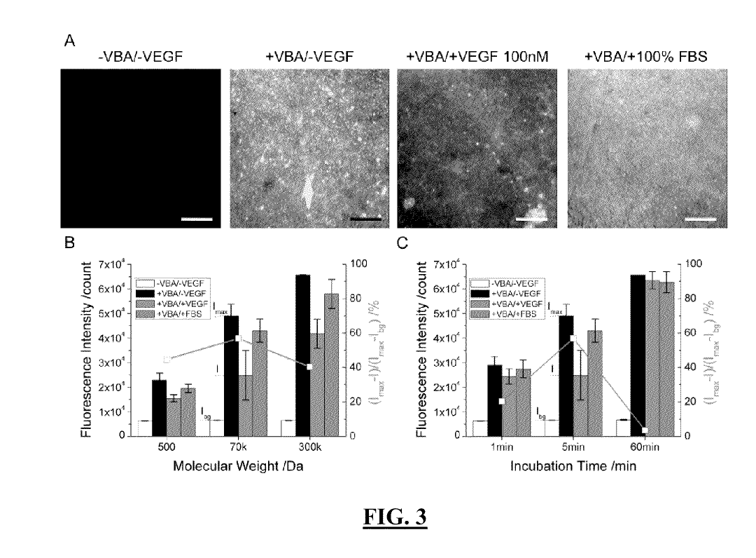

An integrated platform having an optimized signal to noise level was fabricated according to a procedure schematically illustrated in FIG. 3.

In particular, in the illustration of FIG. 3 it is shown the optimization of PLL immobilization by variation of PLL molecular weight and incubation time.

PLL includes positively charged amine group, of which strength is modified with the molecular size of PLL. Metal surface covered with PLL obtains positive charge, of which strength is additionally modified with the incubation time. Therefore, the strength of positive charge on the metal surface increases with size and incubation time of PLL. As a result, the intensity of aptasensor increases with size and incubation time of PLL and then saturated at certain conditions. The amount of target is estimated by normalizing signal decrease in presence of target with the original signal in absence of target.

In particular, a platform has been assembled following the procedures and protocols illustrated in Example 1 varying the molecular weight of the PLL adsorbed on the glass surface or the time of incubation of PLL on the surface.

The slide was thoroughly rinsed with acetone and isopropyl alcohol (IPA) sequentially and then modified with amino-terminal group by immersion in APS (10% v/v with IPA) for 10 minutes followed by rinsing with IPA and drying with N₂ gas. A 14 μL GNP solution was spotted on the modified slide (3 mm in diameter) defined by a PDMS membrane and incubated at room temperature for an hour. Unfixed GNPs were removed by rinsing the substrate three times with 10 μL of DI water. PLL in buffer solution was then immobilized on the GNP-substrate by incubating at room temperature. The unbound PLL were subsequently removed via repeated rinsing five times with 10 μL buffer. 10 μL aptamer in buffer at the concentration of 100 nM were immobilized on the PLL-covered GNP surface for 1 hour above melting temperature (i.e. 70° C.). The unbound aptamers were subsequently removed via rinsing five times with 10 μL of buffer solution. The areas presenting aptasensors, and the PLL conditions were varied as will be apparent to a skilled person upon reading of the disclosure.

The fluorescence of the sensors comprised in the various platforms assembled as described above, was detected following addition of VBA and VEGF. In particular, FIG. 3A shows fluorescence images of experiments with different interaction cases under the same PLL conditions: 70 k, 5 min. FIG. 3B and FIG. 3C show quantified experimental results further varying PLL conditions.

The results are illustrated in FIG. 3, indicate that the intensity of fluorescence signals increased with increase of molecular weight and incubation time. However, the signals were saturated with the weight and the time. The amount of target was estimated with the ratio of the signal change in the presence of targets to signal without targets. As a result, the maximized value was obtained by optimizing the size and the time of PLL.

In this sense, optimal signal-to-noise levels were observed when employing PLL with a molecular weight of about 70 kD and an incubation time of approximately 5 min.

Accordingly, the above results support the conclusion that the performance of aptasensor was maximized at molecular weight of PLL, 70 kDa and incubation time of 5 minute.

### Example 3

### Platform with Optimized for Surface Energy and Stability of the Components

Platforms can be treated to elevate the surface energy of the substrate surface to improve detection through plasmon resonance.

In particular treatment can be performed with UV-ozone (UVO), oxygen plasma or other processes identifiable by a skilled person.

The effect of the treatment on the composition immobilized on the substrate surface was evaluated by executing the treatment among procedures for sensor preparation. The slide was thoroughly rinsed with acetone and isopropyl alcohol (IPA) sequentially and then modified with amino-terminal group by immersion in APS (10% v/v with IPA) for 10 minutes followed by rinsing with IPA and drying with N₂ gas. A 14 μL GNP solution was spotted on the modified slide (3 mm in diameter) defined by a PDMS membrane and incubated at room temperature for an hour. Unfixed GNPs were removed by rinsing the substrate three times with 10 μL of DI water. 70 kDa PLL in buffer solution was then immobilized on the GNP-substrate by incubating for 5 minutes at room temperature. The unbound PLL were subsequently removed via repeated rinsing five times with 10 μL buffer. 10 μL aptamer in buffer at the concentration of 100 nM were immobilized on the PLL-covered GNP surface for 1 hour above melting temperature (i.e. 70° C.). The unbound aptamers were subsequently removed via rinsing five times with 10 μL of buffer solution. Bonding step with UVO and oxygen plasma was added next to APS, PLL, and aptamer immobilization steps separately.

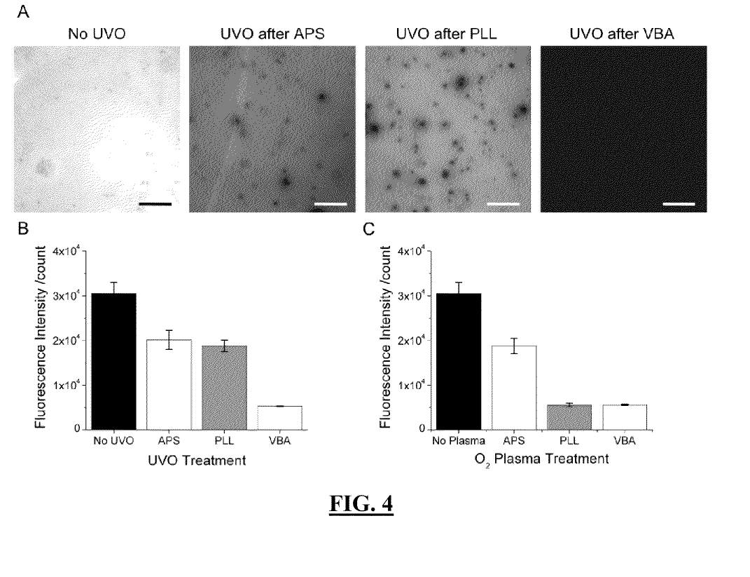

The results illustrated in FIG. 4 indicate that while application of UVO treatment after PLL immobilization resulted in only a small loss in PLL, plasma treatment completely degraded PLL. In particular the UVO treatment partially denatured APS and PLL and completely damaged aptamer. To optimize the platform aptamer immobilization is performed after UVO or plasma treatment for bonding. In particular, based on the above observation, aptamer was immobilized right after UVO treatment for bonding, which still provides a detectable signal sufficient for use of a platform for purposes such as diagnostics.

Conventionally oxygen plasma treatment employs high energy more enough to elevate surface energy for bonding of PDMS and glass. UVO employs relatively low energy to degrade organic molecules on the surface. However, it was found that UVO treatment also elevate surface energy for bonding. Therefore, in this particular example, Applicants used UVO treatment for bonding even though the effectiveness of bonding was relatively lower than oxygen plasma treatment to maintain APS and PLL stable.

### Example 4

### Bonding of Target Producing Material on the Aptasensor Substrate

MCF-7 cells were immobilized on integrated microfluidic platform according to the following procedure.

Device pacification was initially performed as follows. PDMS devices were surface blocked with 5% w/v BSA (Invitrogen, Carlsbad, Calif.) in Tris Buffered Saline with Tween-20 (TBST) for one hour at room temperature before rinsing thrice with MilliQ water.

Cell culturing was performed as follows. Human breast cancer cell line MCF-7 were then cultured on tissue culture treated plastic dishes kept in an incubator at 37° C., 5% CO₂. The cells were fed DMEM supplemented with 10% FBS (Hyclone, Logan, Utah) and passaged at a 1/15 ratio twice weekly.

Cell Trapping on the platform was performed as follows: MCF-7 cells were trypsinized using 1.5 ml of 0.05% trypsin (Invitrogen), pipetted up and down to break up cell clumps, and trypsin inactivated by the addition of 3.5 ml of DMEM+10% FBS. Cells were diluted to 1E6 cells/ml for experimental use. The PDMS device is pre-vacuumed for 10 min in a vacuum chamber with the outlet blocked. After removal from vacuum chamber, 200 μl of cell solution was dropped in the inlet and allowed to self equilibrate. The outlet was then attached to a pressure pump and allowed to run at −5 mmHg for 20 min to load cells into the traps. The excess cells were flushed with working media (DMEM+10% FBS+1 mM estradiol) before starting constant feeding perfusion of 0.5 μl/min over the entire experimental duration.

Probable concern would be the relatively weaker bonding strength created by UVO treatment compared to oxygen treatment. However, the bonding strength by UVO treatment was measured over 15 Psi, which is good enough to trap target particles under negative pressure-driven flow.

### Example 5

### Target Detection in Solution

Detection of VEGF from a solution circulating within the microfluidic platform was performed according to the experiments exemplified below.

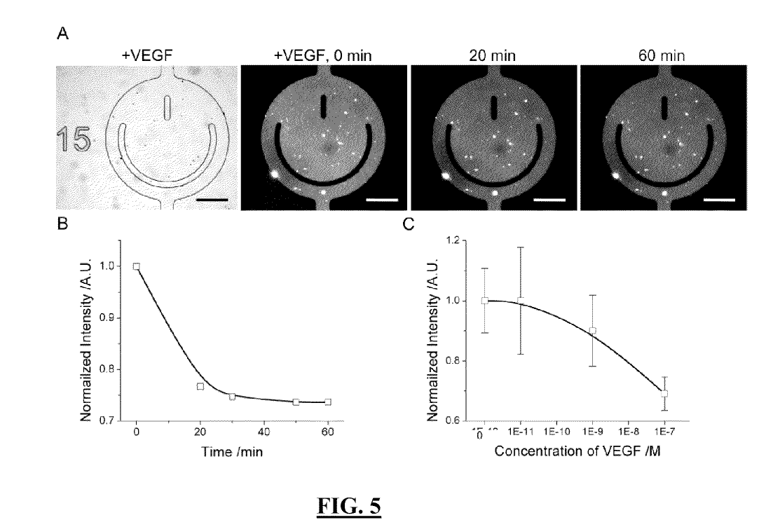

In particular, detection of VEGF at 100 nM was performed within 20 min (FIGS. 5A and 5B) with a limit of detection of 1 nM (FIG. 5C) in buffer solution.

To this extent an aptasensors was prepared and placed homogenously inside microfluidic channels and chambers by flowing aptamer solution after the bonding process (see Example 1). To evaluate dynamic interaction of aptasensor with target, signal from an identical chamber was measured with interaction time, showing that the interaction reached saturation after half an hour. To evaluate the sensitivity of the aptasensor, target of variant concentration was introduced to each separate chamber,

In particular, the glass slide was thoroughly rinsed with acetone and isopropyl alcohol (IPA) sequentially and then modified with amino-terminal group by immersion in APS (10% v/v with IPA) for 10 minutes followed by rinsing with IPA and drying with N₂ gas. A 100 μL GNP solution was spotted on the modified slide (2 cm×3 cm) defined by a PDMS membrane and incubated at room temperature for an hour. Unfixed GNPs were removed by rinsing the substrate three times with 10 μL of DI water. 70 kDa PLL in buffer solution was then immobilized on the GNP-substrate by incubating for 5 minutes at room temperature. The unbound PLL were subsequently removed via repeated rinsing five times with 10 μL buffer. The microfluidic device is then fabricated by replicating structure made of SU-8 on a wafer using PDMS as schematically shown in FIG. 2B. In particular, a mold of SU-8 was fabricated with standard lithography. PDMS microfluidics was fabricated by replicating negative pattern of SU-8. The fabricated PDMS was coated with 5% w/v BSA for 1 hour at room temperature to avoid adsorption of aptamer and target protein to PDMS surface. Both of BSA-coated PDMS microfluidics and PLL-GNP-APS coated glass surface were exposed to UVO for 3 minutes and instantly contact for bonding. The bonded platform was cured more than 1 hour at room temperature for the surface recovery after the UVO treatment. After recovery, an aptamer in buffer at the concentration of 100 nM were immobilized on the PLL-covered GNP surface by perfusion at the rate of 1 uL/min for 1 hour above melting temperature (i.e. 70° C.). The unbound aptamers were subsequently removed via rinsing with buffer of 1 mL. 10 μL VEGF solution with variant concentration replaced buffer solution in the platform and remained for an hour at room temperature. The detached aptamers were subsequently removed via rinsing with buffer of 1 mL.

The related results are illustrated in FIG. 5, which shows VEGF detection in buffer solution in the integrated microfluidic platform.

In particular in the illustration of FIG. 5 representative bright field image before adding VEGF (left) and fluorescent images (middle and right) from the measurement of after 0, 20, and 60 min following the addition of VEGF (FIG. 5A).

In particular, the images show that the signal change was clearly discernable at 1 nM VEGF and barely distinguishable down to 100 μM.

As shown in the charts of FIG. 5B and FIG. 5C the signal decrease upon addition of VEGF reached saturation at 20 minutes and the observed limit-of-detection for VEGF in buffer is 1 nM from measurement of fluorescence signal after 60 min following the addition of VEGF.

Multiplexed detection can be realized by immobilizing several different aptamer complex (aptamer specifically binding to different target and probe emitting light in different wavelengths) on the same metal surface or each aptamer complex on different metal surface in an array format like DNA or protein array. Sample solution containing several targets is dropped on the same metal surface or introduced on different metal surface under guidance of microfluidic channel.

### Example 6

### Target Detection from Immobilized Cells

Detection of VEGF from immobilized cells within the microfluidic platform was performed according to the experiments exemplified below.

In particular, the integrated platform was used to monitor VEGF present in culturing media containing 10% FBS and detect additional VEGF secreted from MCF-7 cells stimulated by estrodiole at 0.1 mM after culturing for 37 hrs.

To this extent the aptasensor was prepared homogenously inside the microfluidic channels and chambers by flowing aptamer solution after bonding process. To evaluate VEGF detection with cells, signals from identical chambers were measured with culturing time, with and without estrodiole, stimulating VEGF secretion pathway. As a result, signal decrease was monitored from both cases up to 22 hrs of culturing time, affected by VEGF presented in 10% FBS culturing media.

In particular, the glass slide was thoroughly rinsed with acetone and isopropyl alcohol (IPA) sequentially and then modified with amino-terminal group by immersion in APS (10% v/v with IPA) for 10 minutes followed by rinsing with IPA and drying with N₂ gas. A 100 μL GNP solution was spotted on the modified slide (2 cm×3 cm) defined by a PDMS membrane and incubated at room temperature for an hour. Unfixed GNPs were removed by rinsing the substrate three times with 10 μL of DI water. 70 kDa PLL in buffer solution was then immobilized on the GNP-substrate by incubating for 5 minutes at room temperature. The unbound PLL were subsequently removed via repeated rinsing five times with 10 μL buffer. The microfluidic device is then fabricated by replicating structure made of SU-8 on a wafer using PDMS as schematically shown in FIG. 2B. In particular, a mold of SU-8 was fabricated with standard lithography. PDMS microfluidics was fabricated by replicating negative pattern of SU-8. The fabricated PDMS was coated with 5% w/v BSA for 1 hour at room temperature to avoid adsorption of aptamer and target protein to PDMS surface. Both of BSA-coated PDMS microfluidics and PLL-GNP-APS coated glass surface were exposed to UVO for 3 minutes and instantly contact for bonding. The bonded platform was cured more than 1 hour at room temperature for the surface recovery after the UVO treatment. After recovery, an aptamer in buffer at the concentration of 100 nM were immobilized on the PLL-covered GNP surface by perfusion at the rate of 1 uL/min for 1 hour above melting temperature (i.e. 70° C.). The unbound aptamers were subsequently removed via rinsing with buffer of 1 mL. Cell Trapping on the platform was performed as follows: MCF-7 cells were trypsinized using 1.5 ml of 0.05% trypsin (Invitrogen), pipetted up and down to break up cell clumps, and trypsin inactivated by the addition of 3.5 ml of DMEM+10% FBS. Cells were diluted to 1E6 cells/ml for experimental use. The PDMS device is pre-vacuumed for 10 min in a vacuum chamber with the outlet blocked. After removal from vacuum chamber, 200 μl of cell solution was dropped in the inlet and allowed to self equilibrate. The outlet was then attached to a pressure pump and allowed to run at −5 mmHg for 20 min to load cells into the traps. The excess cells were flushed with working media (DMEM+10% FBS+1 mM estradiol) before starting constant feeding perfusion in an incubator. A media containing 10% FBS with or without estrodiole is perfused into the platform at the flow rate of 0.5 μL/min in an culturing incubator during experiments.

The platform was extracted from the culturing incubator for detecting fluorescence signals at 0, 12, 22, and 37 hr culturing time.

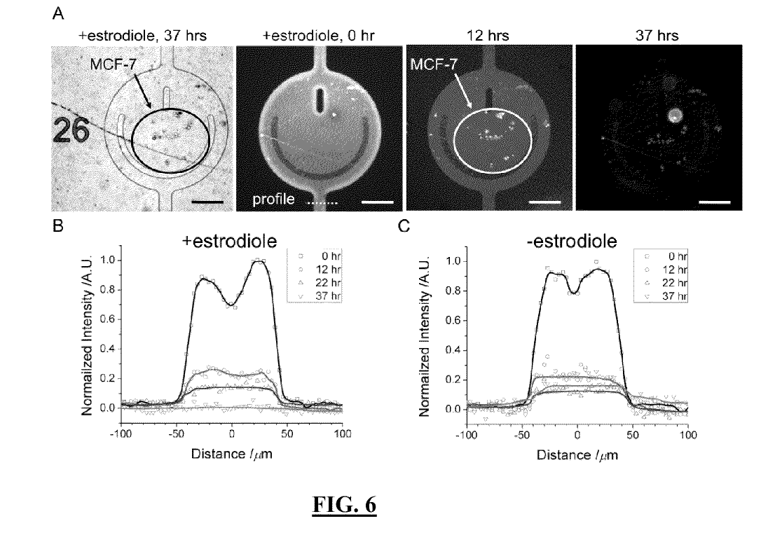

The related results illustrated in FIG. 6 show that the signal completely disappears for cells stimulated by continuous dose of 0.1 mM estradiole for 37 hrs (FIG. 6A and FIG. 6B). In the absence of estradiole, VEGF present in the culture media quickly reduces the fluorescence intensity to a base value, which remains stable over 37 hrs, indicating the absence of additional secreted VEGF from MCF-7 cells (FIG. 6C). However, the complete signal decrease was observed only from the case where the stimulus was introduced, indicating that the estrodiole stimulated additional VEGF secretion from cells.

In summary the above results show that the integrated platform achieved the label-free detection of vascular endothelial growth factor (VEGF) down to 1 nM in buffer solution and also VEGF secreted from MCF-7 (human breast cancer) cells upon continuous stimulation with 0.1 mM estradiole for 37 hrs. Additionally, there was no discernible signal change in the absence of VEGF in buffer or in the absence of the estradiole stimulus in cells.

The above results also supports performance of a multiple detection realized by immobilizing mixture of different aptamer complexes, which comprise for example target binding aptamers specific for different targets each attaching a different probe located on a same spot, as well as aptamer complexes comprising different probes located on one or more spots on different column in the arrayed platform. Additional aptamers sensors and configurations of said aptamers sensors on the platform are identifiable by a skilled person upon reading of the present disclosure.

### Example 7

### Patterning of a Microfluidic Platform

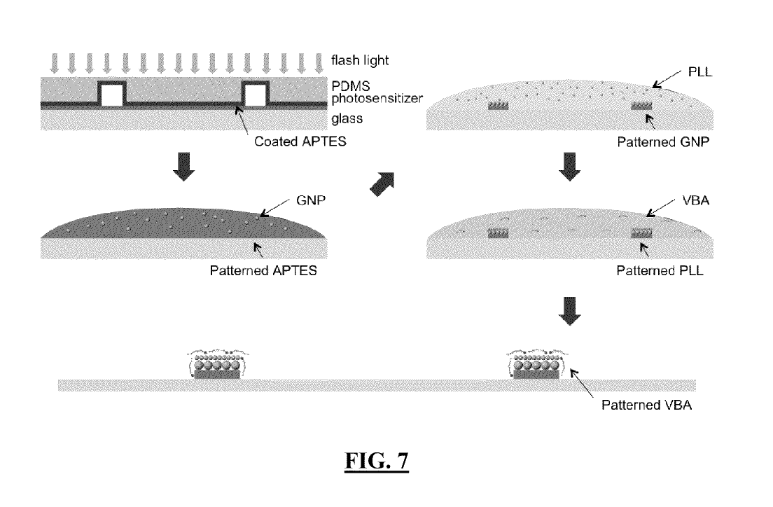

FIG. 7 shows aptasensor Patterning of microfluidic platform assembled according to procedures exemplified in Example 1 was performed based on APS patterning by use of photocatalytic lithography with porphyrins, a type of photosensizer. To this extent, APS was initially coated on the substrate, and then selectively removed by placing a mask coated with photosensitizer in close proximity to the APS coated substrate and then exciting the mask with visible light. This results in the formation of reactive oxygen radicals species being emitted from the photosensitizer and leads to local oxidative decomposition of the APS. The gold nanoparticle (GNP) solution, PLL solution, and aptamer solution are then introduced on the APS-patterned substrate, resulting in selective patterning of aptamer sensor.

Patterning such sensors on the micron or nanometer scale is most feasible via porphyrin-based photocatalytic lithography, although patterning may be performed by traditional lithography, contact printing or dip pen lithography as well. Both of the microfluidic channel and the glass substrate are exposed to UV cleaning treatment to elevate surface energy; subsequently bringing them into contact generates a bond between them. After leaving the bonded platform for an hour at room temperature, aptamer complex solution is introduced and incubated for an hour and then rinsed with buffer solution according to techniques identifiable by a skilled person upon reading of the present disclosure.

### Example 8

### Target Detection Performed with a Patterned Microfluidic Platform

Patterned microfluidic platform assembled using procedures exemplified in Example 7 were used for target detection.

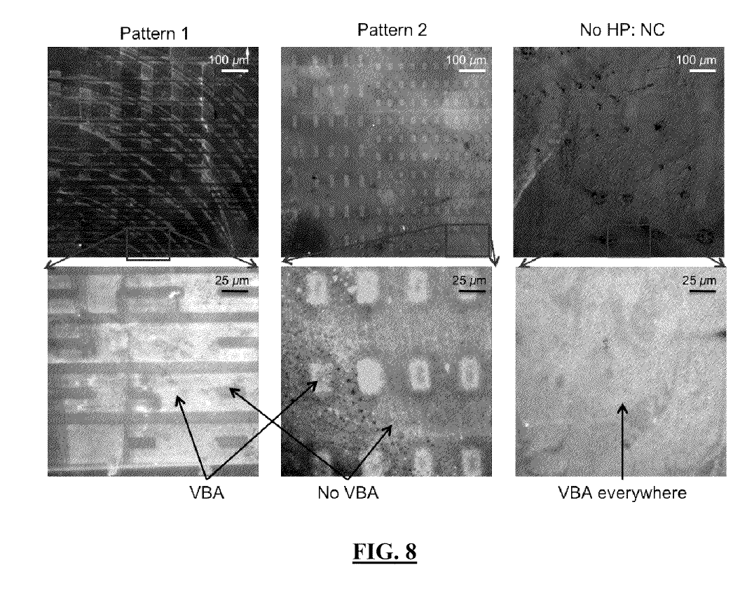

In particular, three patterns were provided that are illustrated in FIG. 8. In particular, pattern 1 was provided by photocatalytically removing APS in the topographical regions defined by squares and lines to provide an aptasensor pattern outside the region where APS was removed. Pattern 2 was provided by photocatalyticaly removing APS outside the topographic region defined by squares, resulting in aptasensor pattern within in the region defined by squares and lines. To provide pattern ‘No HP: NC’ pattern, the slide was treated without removing APS thus resulting in an aptasensor that is patterned on the entire surface.

### Example 9

### Diagnostic Marker Detection Performed Using the Microfluidic Platform

A platform built as described in Example 1 was used to detect VEGF in fluid samples from patients.

The slide was thoroughly rinsed with acetone and isopropyl alcohol (IPA) sequentially and then modified with amino-terminal group by immersion in APS (10% v/v with IPA) for 10 minutes followed by rinsing with IPA and drying with N₂ gas. A 14 μL GNP solution was spotted on the modified slide (3 mm in diameter) defined by a PDMS membrane and incubated at room temperature for an hour. Unfixed GNPs were removed by rinsing the substrate three times with 10 μL of DI water. 70 kDa PLL in buffer solution was then immobilized on the GNP-substrate by incubating for 5 minutes at room temperature. The unbound PLL were subsequently removed via repeated rinsing five times with 10 μL buffer. 10 μL aptamer in buffer at the concentration of 100 nM were immobilized on the PLL-covered GNP surface for 1 hour above melting temperature (i.e. 70° C.). The unbound aptamers were subsequently removed via rinsing five times with 10 μL of buffer solution.

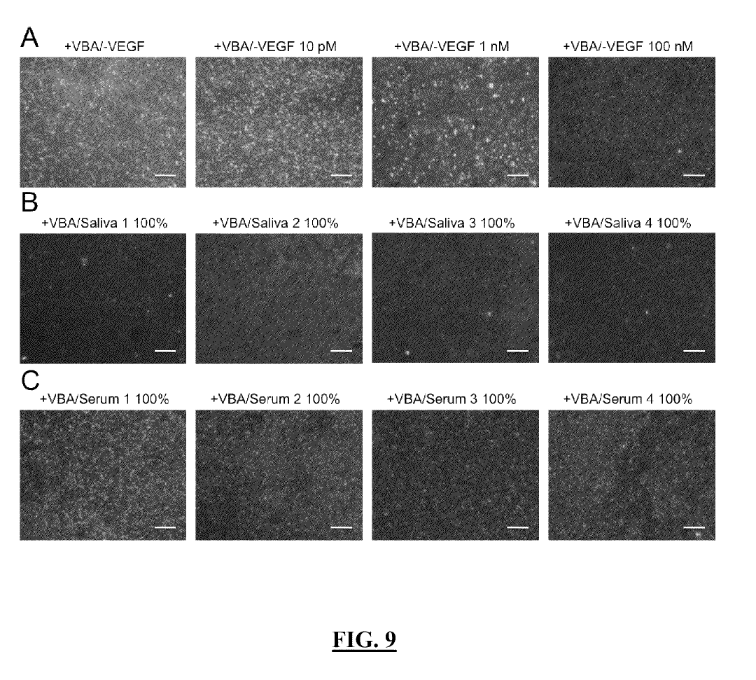

The related results illustrated in FIG. 9 shows that the fluorescent signals decreased with the concentration of VEGF in purified solutions down to 10 pM. B. In saliva samples, the signals decreased corresponding to 100 nM VEGF. With serum samples, the signal decreases corresponding to 10 nM VEGF.

Further quantitative and qualitative detection of VEGF in serum and saliva of patients were performed using the platform herein described.

Qualitatively speaking, the signal decrease indicates presence of target molecules. Quantitatively speaking, relative signal decrease corresponding to the concentration of target molecules. As for the diagnostics, if the amount of target is higher than the level of healthy people, the person that is providing the tested material is suspected to have cancer cells.

The target molecules are secreted from cancer cells and reported to be highly present in biofluids: serum and plasmid. Therefore, the aptasensor can be applicable to diagnostics with biofluids extracted from patients or with sample cells cultured in microfluidics.

The examples set forth above are provided to give those of ordinary skill in the art a complete disclosure and description of how to make and use the embodiments of the, platform aptamers, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The entire disclosure of each document cited (including patents, patent applications, journal articles, abstracts, laboratory manuals, books, or other disclosures) in the Background, Summary, Detailed Description, and Examples is hereby incorporated herein by reference.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

Although any methods and materials similar or equivalent to those described herein can be used in the practice for testing platform aptamers and related systems and methods of the disclosure, specific examples of appropriate materials and methods are described herein for guidance purpose.

A number of embodiments of the disclosure have been described. Nevertheless, it will be understood that various modifications may be made without departing from the spirit and scope of the present disclosure. Accordingly, other embodiments are within the scope of the following claims.

### REFERENCES

1. Ellington, A. D.; Szostak, J. W. Nature 1990, 346, 818-822
2. Tuerk, C.; Gold, L. Science 1990, 249, 505-510
3. Jayasena, S. D. Clin. Chem. 1999, 45, 1628-1650.
4. Sullenger, B. A.; Gilboa, E. Nat. Rev. 2002, 418, 252-258
5. Pestourie, C.; Tavitian, B.; Duconge, F. Biochimie 2005, 87, 921-930
6. Bunka, D. H. J.; Stockley, P. G. Nat. Rev. Microbio. 2006, 4, 588-596
7. Klussmann, S. The Aptamer Handbook: Functional Oligonucleotides and Their Applications; WILEY-VCH Verlag GmbH & Co. KGaA: New York, 2006.
8. Fischer, N. O.; Tarasow, T. M.; Tok, J. B. H. Curr. Opin. Chem. Biol. 2007, 11, 316-328
9. Szakacs, G.; Paterson, J. K.; Ludwig, J. A.; Booth-Genthe, C.; Gottesman, M. M. Nat. Rev. Drug Discovery 2006, 5, 219-234
10. Xiao, Y.; Piorek, B. D.; Plaxco, K. W.; Heeger, A. J. J. Am. Chem. Soc. 2005, 127, 17990-17991
11. Baker, B. R.; Lai, R. Y.; Wood, M. S.; Doctor, E. H.; Heeger, A. J.; Plaxco, K. W. J. Am. Chem. Soc. 2006, 128, 3138-3139
12. Stojanovic, M. N.; Landry, D. W. J. Am. Chem. Soc. 2002, 124, 9678-9679
13. Huang, C. C.; Huang, Y. F.; Cao, Z.; Tan, W.; Chang, H. T. Anal. Chem. 2005, 77, 5735-5741
14. Liu, J.; Lu, Y. Nat. Protocols 2006, 1, 246-252
15. Bagalkot, V.; Zhang, L.; Levy-Nissenbaum, E.; Jon, S.; Kantoff, P. W.; Langer, R.; Farokhzad, O. C. Nano Lett. 2007, 7, 3065-3070
16. Xiao, Y.; Lubin, A. A.; Heeger, A. J.; Plaxco, K. W. Angew. Chem. Int. Ed. 2005, 44, 5456
17. Radi, A. E.; AceroSanchez, J. L.; Baldrich, E.; O′Sullivan, C. K. J. Am. Chem. Soc. 2006, 128, 117-124
18. Wei, H.; Li, B.; Li, J.; Wang, E.; Dong, S. Chem. Commun. 2007, 3735-3737
19. Fang, X.; Sen, A.; Vicens, M.; Tan, W. ChemBioChem 2003, 4, 829-834
20. Shlyahovsky, B.; Li, D.; Weizmann, Y.; Nowarski, R.; Kotler, M.; Willner, I. J. Am. Chem. Soc. 2007, 129, 3814-3815
21. Wang, Y.; Wei, H.; Li, B.; Ren, W.; Guo, S.; Dong, S.; Wang, E. Chem. Commun. 2007, 5220-5222
22. Chen, J.-W.; Liu, X.-P.; Feng, K.-J.; Liang, Y.; Jiang, J.-H.; Shen, G.-L.; Yu, R.-Q. Biosens. Bioelectron 2008, 24, 66-71
23. Li, H.; Rothberg, L. Proc. Natl. Acad. Sci. U.S.A. 2004, 101, 14036-14039
24. Li, H.; Rothberg, L. J. Anal. Chem. 2004, 76, 5414-5417
25. Bock, L. C.; Griffin, L. C.; Latham, J. A.; Vermaas, E. H.; Toole, J. J. Nature 1992, 355, 564-566
26. Vairamani, M.; Gross, M. L. J. Am. Chem. Soc. 2003, 125, 42-43
27. Yau, H. C. M.; Chan, H. L.; Sui, S. f.; Yang, M. Thin Solid Films 2002, 413, 218-223
28. Herne, T. M.; Tarlov, M. J. J. Am. Chem. Soc. 1997, 119, 8916-8920
29. Gearheart, L. A.; Ploehn, H. J.; Murphy, C. J. J. Phys. Chem. B 2001, 105, 12609-12615
30. Nicolai, S. H. A.; Rubim, J. C. Langmuir 2003, 19, 4291-4294
31. Stokes, R. J.; Macaskill, A.; Lundahl, P. J.; Smith, W. E.; Faulds, K.; Graham, D. Small 2007, 3, 1593-1601
32. Bell, S. E. J.; Sirimuthu, N. M. S. J. Phys. Chem. 2005, 109, 7405-7410
33. Weitz, D. A.; Oliveria, M. Phys. Rev. Lett. 1984, 52, 1433
34. Choi, D.; Kang, T. K.; Cho, H.; Choi, Y.; Lee, L. P. Lab on a Chip 2009, 9, 239-243
35. Nicolai, S. H. d. A.; Rodrigues, P. R. P.; Agostinho, S. M. L.; Rubim, J. C. J. Electroanal. Chem. 2002, 527, 103-111
36. Ruckman, J.; Green, L. S.; Beeson, J.; Waugh, S.; Gillette, W. L.; Henninger, D. D.; Claesson-Welsh, L.; Janjic, N. J. Biol. Chem. 1998, 273, 20556-20567
37. Hering Katharina Cialla Dana, Ackermann Katrin, Dörfer Thomas, Möller Robert, Schneidewind Henrik, Mattheis Roland, Fritzsche Wolfgang, Rösch Petra, Popp Jürgen in Anal Bioanal Chem (2008) 390:113-124

What is claimed is:

1. A microfluidic platform comprising one or more chambers, each chamber configured to provide independent conditions therein, each chamber comprising a substrate presenting aptamer sensors.

2. The microfluidic platform of claim 1, wherein each aptamer sensor comprises an aptamer attaching a spectroscopic probe.

3. The microfluidic platform of claim 1, wherein the substrate is a glass substrate.

4. The microfluidic platform of claim 1, wherein the aptamer sensors are physisorbed on surfaces of golden nanoparticles.

5. The microfluidic platform of claim 1, wherein at least one of the one or more chambers is configured to provide a predetermined environment specific for a target producing material.

6. The microfluidic platform of claim 1, wherein the substrate of at least one of the one or more chambers is patterned.

7. A method to fabricate a chamber of a microfluidic platform, the method comprising
depositing carriers on a suitable substrate, the carriers adapted to attach aptamers, thus forming a carriers-substrate combination;
bonding the carriers-substrate combination to a microfluidic structure open at its bottom, the binders-substrate combination forming a bottom surface thus resulting in a microfluidic chamber;
introducing the aptamers into the microfluidic chamber; and
attaching the aptamers to the carriers in the microfluidic chamber.

8. The method of claim 7, wherein depositing is performed by depositing golden nanoparticles (GNP) and poly-L-lysine (PLL) on an APS-coated glass substrate, and wherein the carriers-substrate combination is a PLL-GNP-APS-glass combination.

9. The method of claim 8 wherein introducing the aptamers is performed by flowing a solution containing aptamers into the microfluidic chamber.

10. The method of claim 8, wherein attaching the aptamers to the carriers in the chamber is performed by physisorbing the aptamers to the GNP.

11. The method of claim 7, wherein the bonding is UVO bonding.

12. A method to detect targets from a target providing material, the method comprising
providing a microfluidic chamber comprising a substrate on which a target binding aptamer attaching a spectroscopic probe is located, the target binding aptamer capable of specifically binding a pre-determined target;
placing one or more target producing materials in the microfluidic chamber on the substrate;
detecting a first spectrum of the spectroscopic probe attached to the target binding aptamer;
stimulating the target producing material for a time and under conditions to allow production of the target from the target producing material and binding of the target with the target binding aptamer;
detecting a second spectrum of the spectroscopic probe attached to the target binding aptamer following binding of the target with the aptamer; and
comparing the first spectrum and the second spectrum.

13. A detection method comprising:
providing a microfluidic chamber comprising a substrate on which aptamer sensors are located on carriers, each aptamer sensor comprising an aptamer attaching a spectroscopic probe, the aptamer capable to specifically bind a target;
placing one or more cells in the microfluidic chamber above the substrate;
stimulating the one or more cells to elicit generation of targets from the one or more cells, the targets suitable to be detected by at least one aptamer sensor, the aptamers capable to leave the carriers when the aptamer sensors comprise aptamers specific to the generated targets;
exciting the spectroscopic probe; and
detecting a signal from the spectroscopic probe dependent on an amount of aptamer sensors located on the carriers after the stimulating.
